(19) United States
(12) Patent Application Publication
(10) Pub. No.: US 2007/0179488 A1
(43) Pub. Date: Aug. 2, 2007

(54) Diffuser assembly for controlling a light intensity profile

(76) Inventors: Robert M. Trusty, Cincinnati, OH (US); Eric B. Smith, Cincinnati, OH (US)

Correspondence Address: Christopher W. Elswick, 2000 Courthouse Plaza, NE, 10 West Second Street, Dayton, OH, 45402-1758, US

(21) Appl. No.: 11/346,039

(22) Filed: Feb. 2, 2006

(51) Int. Cl. A61B 18/18

(52) U.S. Cl. 606/16

(57) ABSTRACT

A diffusive tip assembly for controlling a light intensity profile during a medical procedure is provided. The diffusive tip assembly includes an optical fiber including a core and a diffuser tip that surrounds a distal end of the core. The diffuser tip includes multiple light directing features that are shaped and arranged in a pattern along an axial length of the diffuser tip. The light directing features are capable of directing light traveling from the core in a radial direction toward an outer surface of the diffuser tip for irradiating a material in proximity of the diffuser tip. An optical coupling material is disposed between the core and the diffuser tip. The optical coupling material and surfaces of the light directing features form boundaries upon which light rays traveling from the core are incident. The pattern of light directing features has a proximal portion, a central portion and a distal portion where a dimensional property of light directing features located at the proximal and distal regions is different than the dimensional property of light directing features located at the central region. The dimensional property is selected to provide a flattened light intensity profile during use.

### FIELD OF THE INVENTION

The present application relates generally to a diffuser assembly for controlling a light intensity profile.

### BACKGROUND OF THE INVENTION

Surgeons frequently employ medical instruments which incorporate laser technology in the treatment of benign prostatic hyperplasia, commonly referred to as BPH. BPH is a condition of an enlarged prostate gland, in which the gland having BPH typically increases in size to between about two to four times from normal. Lasers are used to treat BPH in two different ways. Lasers that reduce prostate volume through surface ablation of tissue introduce a concentrated beam of light to be absorbed by the tissue surface, typically through a side-firing optical fiber introduced to the urethra through a cystoscope's working channel. Lasers that reduce prostate volume through interstitial laser coagulation (ILC) introduce a diffuse beam of light from within the prostate using a radially emitting diffuser tip fiber inserted through the urethral wall and into the prostatic tissue. Surgeons treating BPH in this second manner must have durable optical fibers that distribute light radially in a predictable and controlled manner, and must also be capable of bending without breaking, whereby small-sized or slender optical fibers offer an additional advantage to the surgeon.

An optical fiber which is adapted to be employed for ILC typically contains a glass core surrounded by cladding, a buffer layer, and an outer alignment sleeve. The cladding protects the inherently weaker glass core by imparting a mechanical support to the core. The cladding necessarily possesses an index of refraction which is lower than that of the core in order to function as an optical waveguide.

An optical fiber with a diffuser portion for diffusing light emitted at an end thereof has been disclosed. The optical fiber leading end can have a diffuser portion formed of a stripped core of a typical optical fiber, an optical coupling layer, and an outer or alignment sleeve. The optical coupling layer, replacing a part of the cladding and the buffer layer of the optical fiber, has an index of refraction matching or exceeding that of the core so as to draw the light out of the core using well-known physical principles. The alignment sleeve may be abraded or roughened, in order to conduct light from the optical coupling layer to the exterior, while heat staking or ultrasonic welding may be used to apply or attach the outer sleeve covering the diffuser tip to a further separate portion of the sleeve located towards the end of the optical fiber.

FIG. 1 illustrates a diffuser tip assembly including a diffuser tip 100 having an abraded internal surface 102. These abrasions 104 are provided such that, when a light ray is transmitted through core 106 and encounters the internal surface 102, the abrasion alters the normal trajectory of the light ray, allowing the light ray to escape the core for transmission to an outer surface 108 of the diffuser tip 100.

As is known in the art, higher angle rays (higher order modes) tend to escape from the more proximal section of the de-cladded core 106 while lower angle rays (lower order modes) tend to escape from the core later or continue to travel through the core and strike a light scattering component 110 where the light is scattered or otherwise redirected. Referring to FIG. 2, this, in some prior art fibers, tends to produce a light intensity profile that has two distinct peaks P₁ and P₂ separated by a valley V. While randomly abrading the internal surface 102 can provide a light intensity profile acceptable for treatment of BPH, it is desirable to provide a light intensity profile that more closely resembles an ideal, flat intensity profile (represented by dotted lines).

### SUMMARY OF THE INVENTION

In an aspect, a diffusive tip assembly for controlling a light intensity profile during a medical procedure is provided. The diffusive tip assembly includes an optical fiber including a core and a diffuser tip that surrounds a distal end of the core. The diffuser tip includes multiple light directing features that are shaped and arranged in a pattern along an axial length of the diffuser tip. The light directing features are capable of directing light traveling from the core in a radial direction toward an outer surface of the diffuser tip for irradiating a material in proximity of the diffuser tip. An optical coupling material is disposed between the core and the diffuser tip. The optical coupling material and surfaces of the light directing features form boundaries upon which light rays traveling from the core are incident. The pattern of light directing features has a proximal portion, a central portion and a distal portion where a dimensional property of light directing features located in at least one of the proximal and distal regions is different than the dimensional property of light directing features located in the central region. The dimensional property is selected to provide a flattened light intensity profile during use.

In another aspect, a method of forming a medical device including a diffusive tip assembly is provided. The method includes locating a core of an optical fiber within a bore defined by an inner surface of a diffuser tip. The diffuser tip is provided with multiple light directing features arranged in a pattern along an axial length of the diffuser tip. The light directing features are capable of directing light traveling from the core in a radial direction toward an outer surface of the diffuser tip for irradiating a material in proximity of the diffuser tip. The pattern of light directing features have a proximal portion, a central portion and a distal portion where a dimensional property of light directing features located in at least one of the proximal and distal regions is different than the dimensional property of light directing features located in the central region. The dimensional property being selected to provide a flattened light intensity profile during use.

The details of one or more embodiments of the invention are set forth in the accompanying drawings and the description below. Other features, objects, and advantages of the invention will be apparent from the description and the drawings, and from the claims.

### BRIEF DESCRIPTION OF THE FIGURES

FIG. 1 is an unscaled, diagrammatic section view of a prior art diffuser tip;

FIG. 2 is an illustrative plot of light flux density relative to distance along the light diffuser tip of FIG. 1;

FIG. 3 is a schematic view of an embodiment of a medical device;

FIG. 4 illustrates a diagrammatic, perspective view of an embodiment of an optical fiber assembly;

FIGS. 5 and 5A are unscaled, diagrammatic section and detailed views of an embodiment of a diffusive tip assembly for use with the medical device of FIG. 1;

FIG. 6 is an unscaled, diagrammatic section view of another embodiment of a diffusive tip assembly for use with the medical device of FIG. 1 that includes a pattern of light directing features having a variable pattern periodicity;

FIG. 7 is an unscaled, diagrammatic section view of another embodiment of a diffusive tip assembly for use with the medical device of FIG. 1 that includes a pattern of light directing features having a variable pattern depth;

FIG. 8 is an unscaled, diagrammatic section view of another embodiment of a diffusive tip assembly for use with the medical device of FIG. 1 that includes a pattern of light directing features having a variable pattern periodicity;

FIG. 9 is an unscaled, diagrammatic section view of another embodiment of a diffusive tip assembly for use with the medical device of FIG. 1 that includes a pattern of light directing features having variable pattern groove and land widths;

FIGS. 10 and 10A are unscaled, diagrammatic section and detail views of another embodiment of a diffusive tip assembly for use with the medical device of FIG. 1 that includes a pattern of light directing features having a variable pattern of wall angles;

FIGS. 11 and 11A are unscaled, diagrammatic section and detail views of another embodiment of a diffusive tip assembly for use with the medical device of FIG. 1 that includes a pattern of light directing features having a variable pattern of bottom angles;

FIG. 12 is an unscaled, diagrammatic section view of another embodiment of a diffusive tip assembly for use with the medical device of FIG. 1; and

FIG. 13 is an illustrative ideal plot of light flux density relative to distance along a light diffusing tip having a flattened light intensity profile.

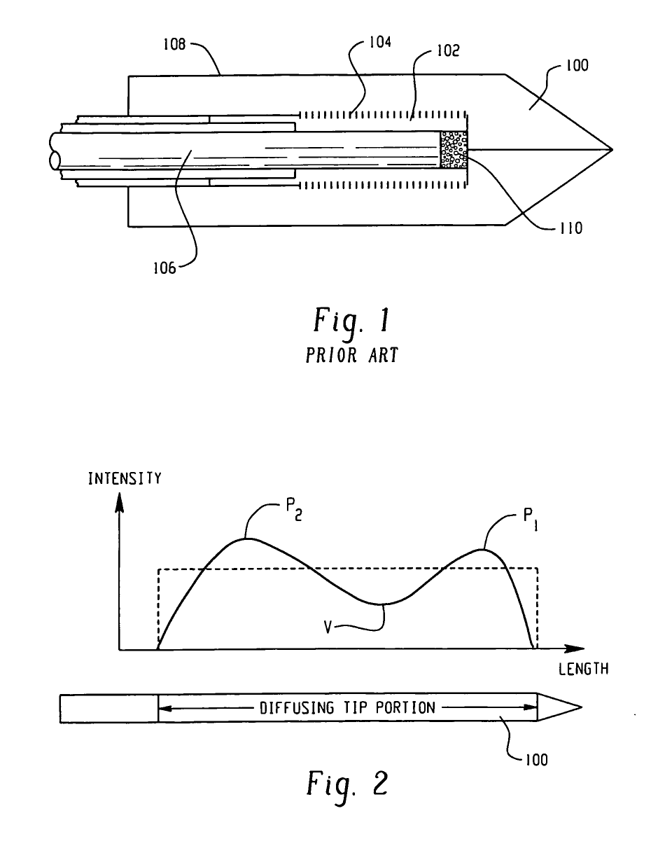

### DETAILED DESCRIPTION OF THE INVENTION

As used herein, the term “proximal” refers to a location on a medical device 10 or a component thereof that is closer to a source of light energy and the term “distal” refers to a location on the medical device or a component thereof that is farther from the source of light energy. Typically, the source of light energy of the medical device 10 is located outside a patient's body and the distal end of the medical device is insertable into the patient's body for a surgical procedure.

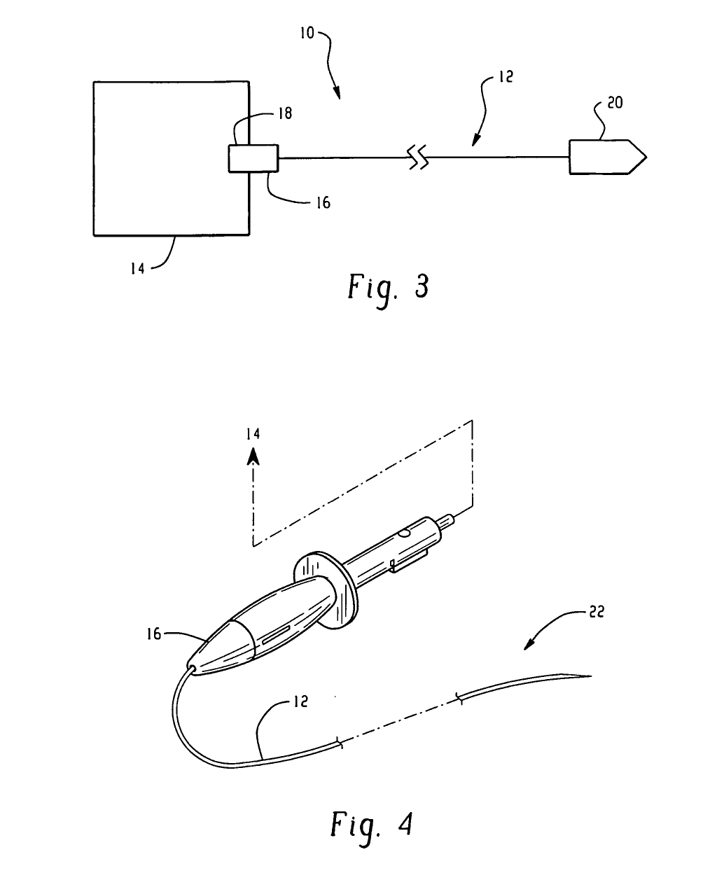

FIG. 3 shows the exemplary medical device 10 for diffusing light from an optical fiber 12, for example, for treatment of BPH. The medical device 10 includes the source of light energy 14, preferably a laser. The optical fiber 12 is connected to the source of light energy 14 through an intermediary connector 16 at the proximal end of the fiber, which is attached to a connection port 18 of the source. A diffuser portion 20 is provided at the distal end of the optical fiber 12. An exemplary connector 16 and connection port 18 are described in U.S. Pat. No. 5,802,229 issued to Evans et al., the details of which are hereby incorporated by reference as if fully set forth herein. In some embodiments, the optical fiber 12 is provided and sold separately from the source of light energy 14, as an optical fiber assembly 22, as represented by FIG. 4.

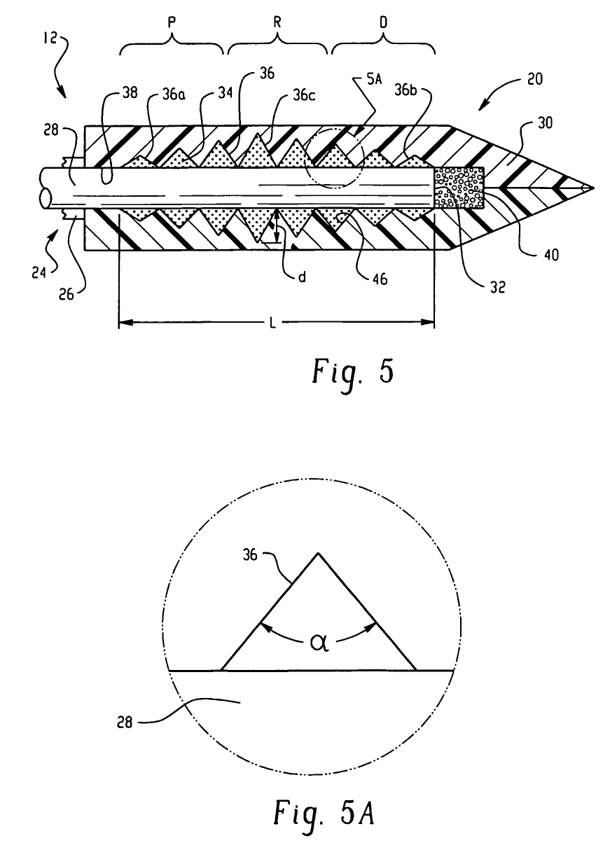

Referring now to FIG. 5, optical fiber 12 includes diffuser portion 20 and a light transmitting portion 24. At the light transmitting portion 24, a cladding 26 surrounds the core 28. In some embodiments, a sleeve (not shown) may also surround the cladding 26 and the core 28. Core 28 may be formed, for example, of silica glass and the material used to form the cladding 26 has an index of refraction that is lower than an index of refraction of the core 28 material so as to contain the light within the core. Cladding 26 terminates at a proximal end of a diffuser tip 30 and the core 28 extends into the diffuser tip of the diffuser portion 20 and terminates at a distal end 32. Diffuser tip 30 may be composed of a material that is flexible, is non-absorbent of laser energy in the wavelengths of interest, has a high melt temperature and is optically diffusing. Suitable materials for forming the diffuser tip 30 include perfluoroalkoxy (PFA) impregnated with barium sulfate, where the barium sulfate assists in scattering light energy, ethylenetetraflouroethylene (ETFE) and other types of flouropolymers.

The distal portion of the core 28 extending into the diffuser tip 30 is used to deliver light to the diffuser portion 20 of the fiber 12 and is surrounded by an optical coupling material 34 at least partially disposed within a series of light directing features 36 that extend outwardly relative to a central, longitudinal axis of the diffuser tip 30. The illustrated features 36 take the form of triangular recesses or grooves at the inner surface of the tip 30. More generally, the light directing features 36 are sized, shaped and/or arranged in a predetermined pattern that is selected to provide a desired light intensity profile as will be described in greater detail below. The optical coupling material 34 is a material having an index of refraction that is the same or higher than the index of refraction of the core 28 and the diffuser tip 30. Any suitable optical coupling material may be employed, such as XE5844 Silicone, which is made by General Electric Company; UV50 Adhesive, available from Chemence, Incorporated in Alpharetta, Ga.; and, 144-M medical adhesive, which is available from Dymax of Torrington, Conn.

A light-scattering component 40, which is filled with a light-scattering material and located at the distal end 32 of the core 28, can reflect light back into the core so as to provide a more even or uniform light distribution. Alexandrite, for example, can be employed as a light-scattering material for component 40. Other suitable light scattering materials include aluminum oxide, titanium dioxide and diamond powder. In addition to its light-scattering properties, the light-scattering component 40 material can, in some embodiments, fluoresce in a temperature-dependent manner upon being stimulated by light, with this property adapted to be used to measure temperature in tissue in proximity to the diffuser tip 30. In some embodiments, optical coupling adhesive, such as that described above, can be used to suspend the light-scattering materials such as alexandrite particles therein and can serve as the base material for the light-scattering component 40. A method of forming various optical fiber 12 components including a light scattering component 40 can be found in U.S. Pat. No. 6,718,089 issued to James, IV et al. and U.S. patent application Ser. No. 10/741,393, entitled “Optical Fiber Tip Diffuser and Method of Making Same”, the details of both of which are hereby incorporated by reference as if fully set forth herein.

Referring still to FIG. 5, the light directing features 36 extend along an axial length L of the diffuser tip 30 from a proximal-most light directing feature 36a to a distal-most light directing feature 36b. Each light directing feature 36 is formed as a shaped recess extending outwardly from the inner surface 38 of the diffuser tip 30 that is filled with the optical coupling material 34 disposed between the core 28 and the diffuser tip. The light directing features 36 extend about a periphery of the core 28 as a set of spaced-apart rings, which are triangular in the case of FIG. 5. Other configurations are possible, however. For example, the light directing features 36 can be formed as a continuous, e.g., spiral, thread-like recess extending about the core 28.

Light is typically launched into the fiber 12 in an angular distribution that can be contained by a waveguide. At the distal portion of the core 28 where the cladding 26 is removed, the angular distribution of the light can cause the light rays to escape in a distribution along the length of the diffuser tip 30. As mentioned above, higher angle rays (higher order modes) tend to attempt escape from the core 28 the earliest while lower angle rays (lower order modes) tend to attempt escape from the core 28 later or continue to travel the core 28 to the end 32 and strike the light scattering component 40 where the light is scattered or otherwise redirected.

The light directing features 36 are formed in a predetermined pattern that is selected to provide a flattened light intensity profile. More particularly, the light directing features 36 located at a central region R have a dimensional property (e.g., such as recess depth) that is selected to scatter the light with greater efficiency than the light directing features 36 located at proximal region P and distal region D. This dimensional property at R is different from (e.g., less than or greater than) the same dimensional property of light directing features 36 located at proximal and distal regions P and D, which can increase the relative probability, as between R and regions P and D outside of R, that light rays incident on boundaries 46 at R are at an angle of incidence that is less than an associated critical angle. The critical angle is the smallest angle of incidence at which total internal reflection occurs and is determined using Snell's Law
n₁sin(θ₁)=n₂sin(θ₂)
where,
n₁ is the index of refraction of the optical coupling layer,
n₂ is the index of refraction of the diffuser tip material,
θ₁ is the angle of incidence, and
θ₂ is the angle of refraction.

The critical angle case is where the angle of refraction θ₂ is 90 degrees with respect to the normal of the boundary. By increasing the probability that light rays impact boundaries 46 between the optical coupling material 34 and the diffuiser tip 30 material at an angle less than the critical angle within the central region R, increased radial light scatter can be realized along the central region. Conversely, by decreasing the probability that light rays in proximal and distal regions P and D outside of R impact boundaries at an angle less than the critical angle, light will be coupled away from P and D. The net effect is expected to be a redistribution of peak energy to the valley region of FIG. 2 and the desired flattening of the intensity profile.

The light directing features 36 form a pattern of geometric shapes that can pull light out of the core 28 at a relatively even rate along L to provide a more even or flattened light intensity profile, for example, notwithstanding the tendencies described above. In the embodiment illustrated by FIG. 5, the light directing features 36 are in the shape of triangular recesses and are arranged in a substantially constant periodic pattern. Each light directing feature 36 has an apex angle α and a depth d. The depths d of light directing features 36 increase from the end light directing features 36a and 36b to the light directing feature 36c located between the end light directing features 36a, 36b. Additionally, the apex angles a (FIG. 5A) decrease from the end light directing features 36a, 36b to the centrally located light directing feature 36c. By providing P and D with light directing features 36 having a greater α, angles of incidence of light incident on boundaries 46 within P and D will be more often greater than the respective critical angles, which will redistribute some of the light back through the core 28 to light directing features at R. The light directing features 36 at R, having a smaller α, present their boundaries 46 such that the light incident thereon tend to have an angle of incidence less than their respective critical angles which allows light to pass through the boundary and toward outer surface 45.

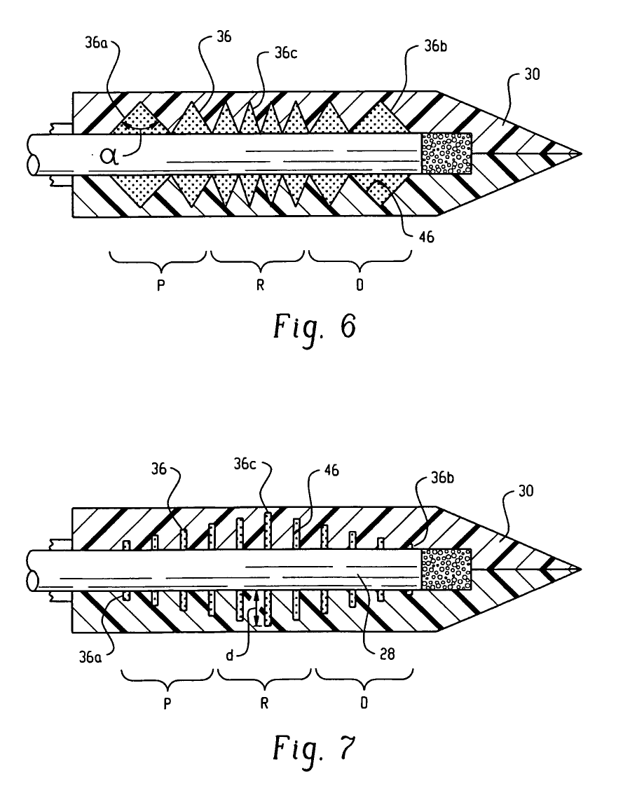

Referring now to FIG. 6, the light directing features 36 are in the shape of triangular recesses and are arranged in a varying periodic pattern that increases from the end light directing features 36a and 36b toward the center light directing feature 36c. Each light directing feature 36 has an apex angle α and substantially the same depth d. The apex angles α decrease from the end light directing features 36a, 36b to the centrally located light directing feature 36c. This varying periodicity causes higher order modes incident on boundaries 46 at P and lower order modes incident on boundaries 46 at D to transfer to R in a fashion similar to that described with reference to FIG. 5.

Any other suitable recess shape and arrangement can be utilized that provides a pattern resulting in a flattened light intensity profile. For example, referring to FIG. 7, the depths d of the light directing features 36 in the form of rectangular recesses increase from the end light directing features 36a, 36b to the centrally located light directing feature 36c. In this embodiment, the boundaries 46 are presented at a constant angle along L, but the cross-sectional area that can interact with the light is reduced at P and D, encouraging the light to exit the core 28 at R.

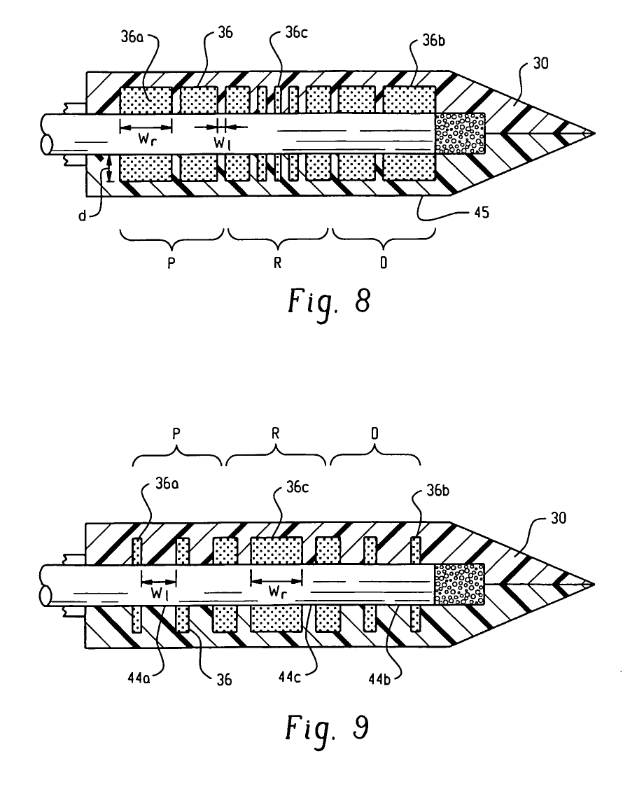

Referring to FIG. 8, light directing features 36 in the form of rectangular-shaped recesses may also be shaped and arranged in a pattern of variable periodicity where diffuser tip 30 includes light directing features 36 in the shape of rectangular recesses having a width Wᵣ that are separated by lands 44 having a width Wₗ. The recesses have widths Wᵣ that decrease from end light directing features 36a and 36_{b} to centrally located light directing feature 36c such that the widths Wᵣ of light directing features 36 within a central region R are less than at regions P and D. It should be noted that the light directing features 36 typically have a depth d that is much less than their respective width Wᵣ. In some embodiments, the ratio of Wᵣ to d is about 3:1 or more, such as about 5:1 or more, such as about 8:1 or more such as 10:1 or more, such as between about 3:1 and 10:1. At these shallow depths in the embodiment of FIG. 8, the relatively wide light directing features 36 at P and D are relatively few due to the lower frequency in those regions, encouraging light to travel to R, while the relatively narrow light directing features of greater frequency at R collectively present more surface for the light incident thereon tend to have an angle of incidence less than their respective critical angles which allows light to pass through the boundary and toward outer surface 45.

FIG. 9 illustrates another example where the relatively wide lands between adjacent light directing features 36 at P and D encourage light to travel to R where the lands are narrower. The pattern of light directing features of FIG. 9 has a fixed period, however, the period may vary.

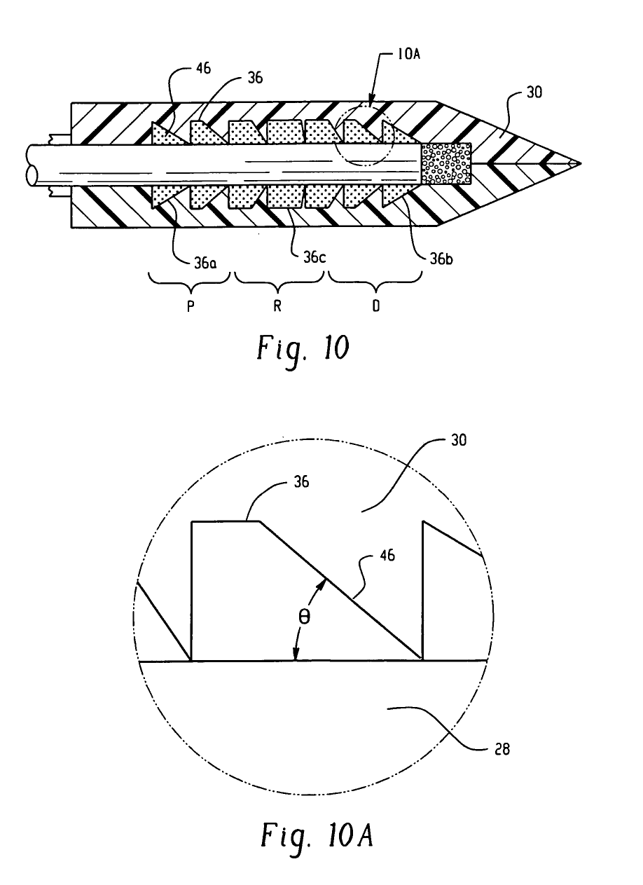

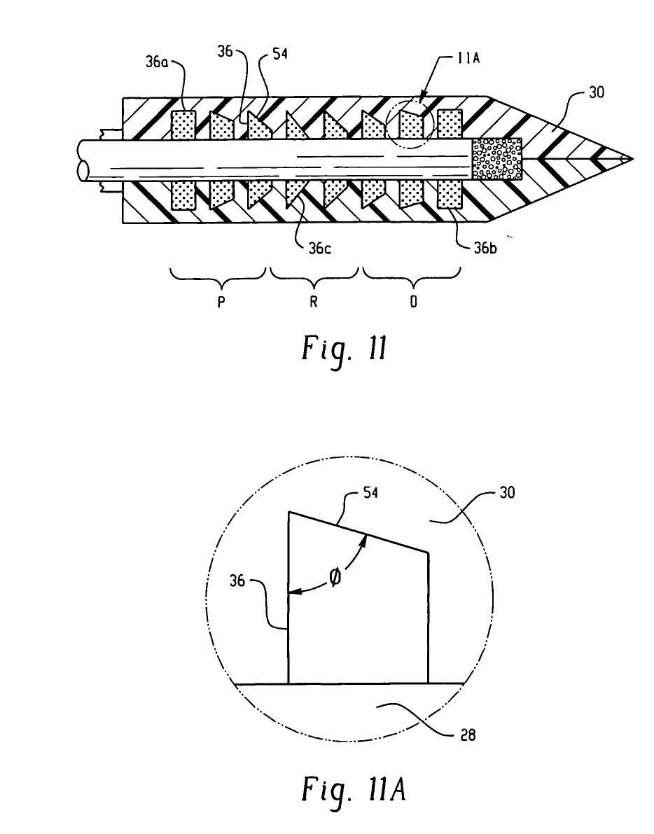

Referring now to FIGS. 10 and 10A, light directing features 36 are each of substantially constant width Wᵣ and form a pattern of variable wall 46 angles θ. The angles θ increase from the end light directing features 36a and 36b to the centrally located light directing feature 36c such that the angles θ of the light directing features 36 within central region R are greater than those at regions P and D. In another embodiment, FIGS. 11 and 11A show light directing features 36 in a pattern of variable groove bottom 54 angles φ. The angles φ decrease from the end light directing features 36a and 36b to the centrally located light directing feature 36c such that the angles φ of the light directing features 36 within central region R are less than those at regions P and D.

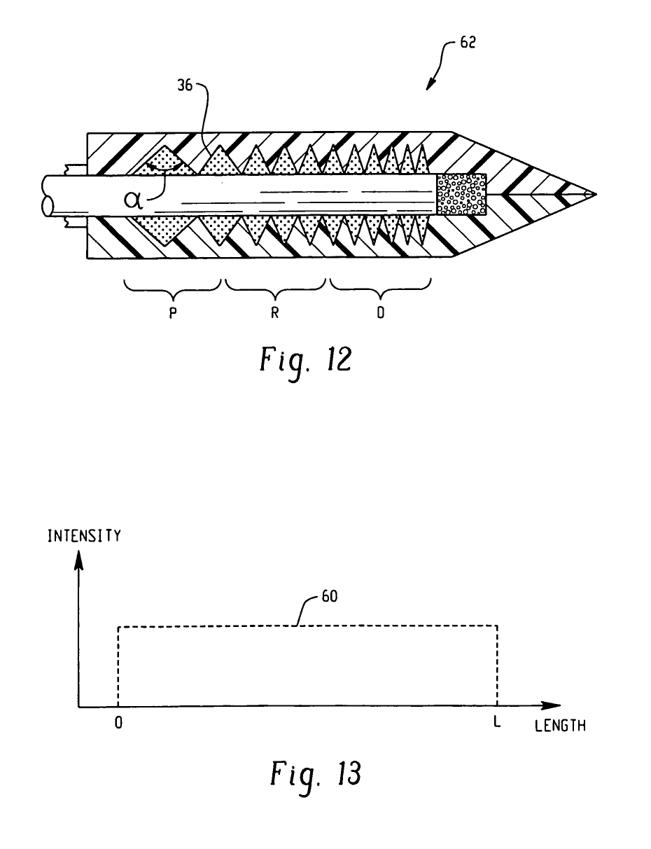

In some embodiments, a dimensional property, such as any of those described above, of the light directing features 36 in only one of P or D may be different than the dimensional property of the light directing features in R. For example, FIG. 12 shows another embodiment of a diffuser tip assembly 62 including light directing features 36 in the shape of triangular recesses. The light directing features 36 are in a pattern where a dimensional property of light directing features in only P is different than the dimensional property of light directing features in R to direct light from P toward R. In this embodiment, the frequency of the light directing features 36 in R and D is greater than in P. In some embodiments, the frequency of the light directing features 36 along both R and D is substantially constant. Additionally, the apex angles α are less within both R and D than within P, with each light directing feature 36 having about the same α in both R and D.

The light directing features 36 of the various embodiments described above may be formed by any suitable and controlled method, such as by molding the light directing features along with the diffuser tip 30. In some embodiments, the light directing features 36 are cold formed by pressing a suitably-shaped tool or tools such as a forming wire or ring into the inner surface 38 of the diffuser tip 30 and then removing the tool. P and D may be about, for example, 20 to 40 percent of L while R may be about 20 to 60 percent of L. L may be about one centimeter, for example.

The above-described patterns of light directing features 36 are sized, shaped and/or arranged to increase the probability within the central region R (e.g., compared to regions P and/or D) that light rays traveling from the optical fiber 12 toward the diffuser tip 30 are incident on surfaces of the light directing features 36 at an angle of incidence that is less than an associated critical angle to allow for refraction of at least a portion of the light rays through the diffuser tip. Referring to FIG. 13, this can provide a desired flattened light intensity profile (e.g., without a valley) that is closer to an ideal flattened light intensity profile 60 by redirecting light rays from regions P and/or D toward the central region R. In some embodiments, the light intensity along region L of the light intensity profile will be no less than about 80 percent (e.g., no less than about 85 percent, no less than about 90 percent) of a peak or maximum intensity.

A number of detailed embodiments have been described. Nevertheless, it will be understood that various modifications may be made. For example, the diffuser tip 30 may be formed as part of a continuous sleeve, for example, that extends along about the entire length of the optical fiber 12. Accordingly, other embodiments are within the scope of the following claims.

What is claimed is:

1. A diffusive tip assembly for controlling a light intensity profile during a medical: procedure, the diffusive tip assembly comprising:
an optical fiber including a core;
a diffuser tip that surrounds a distal end of the core, the diffuser tip including multiple light directing features that are shaped and arranged in a pattern along an axial length of the diffuser tip, the light directing features capable of directing light traveling from the core in a radial direction toward an outer surface of the diffuser tip for irradiating a material in proximity of the diffuser tip; and
an optical coupling material disposed between the core and the diffuser tip, the optical coupling material and surfaces of the light directing features forming boundaries upon which light rays traveling from the core are incident;
wherein the pattern of light directing features has a proximal portion, a central portion and a distal portion;
wherein a dimensional property of light directing features located in at least one of the proximal and distal regions is different than the dimensional property of light directing features located in the central region, the dimensional property being selected to provide a flattened light intensity profile during use.

2. The diffusive tip assembly of claim 1, wherein the dimensional property of light directing features located in the proximal and distal regions is different than the dimensional property of light directing features located in the central region

3. The diffusive tip assembly of claim 2, wherein the light directing features are formed as shaped recesses extending radially from an inner surface of the diffuser tip and the dimensional property is recess depth such that recess depths of light directing features located in the proximal and distal regions are different than recess depths of light directing features located in the central region.

4. The diffusive tip assembly of claim 2, wherein the light directing features are formed as shaped recesses extending radially from an inner surface of the diffuser tip and the dimensional property is periodicity of the light directing features such that the periodicity of light directing features located in the proximal and distal regions are different than the periodicity of light directing features located in the central region.

5. The diffusive tip assembly of claim 2, wherein the light directing features are formed as shaped recesses extending radially from an inner surface of the diffuser tip and the dimensional property is axial width of the recesses such that axial widths of light directing features located in the proximal and distal regions are different than axial widths of light directing features located in the central region.

6. The diffusive tip assembly of claim 2, wherein the light directing features are formed as shaped recesses extending radially from an inner surface of the diffuser tip, wherein at least one surface of each shaped recess is disposed at an angle with respect to an elongated axis of the diffuser tip, the angles of the surfaces of light directing features located in the proximal and distal regions being different than the angles of the surfaces of light directing features located in the central region.

7. The diffusive tip assembly of claim 2, wherein the dimensional property of light directing features located in the proximal and distal regions increases relative to light directing features located in the central region.

8. The diffusive tip assembly of claim 1, wherein the light directing features are formed as shaped recesses extending radially from an inner surface of the diffuser tip, one or more of the shaped recesses being triangular in cross-section.

9. The diffusive tip assembly of claim 1, wherein the light directing features are formed as shaped recesses extending radially from an inner surface of the diffuser tip, one or more of the shaped recesses being rectangular in cross-section.

10. The diffusive tip assembly of claim 1, wherein the light directing features are formed by ring-shaped grooves that are spaced-apart axially from each other.

11. The diffusive tip assembly of claim 1, wherein the light directing features are formed by a continuous, spiral groove.

12. The diffusive tip assembly of claim 1, wherein the central region is between about 20 and 60 percent of the axial length.

13. A method of forming a medical device including a diffusive tip assembly, the method comprising:
locating a core of an optical fiber within a bore defined by an inner surface of a diffuser tip; and
providing the diffuser tip with multiple light directing features as shaped recesses arranged in a pattern along an axial length of the diffuser tip, the light directing features capable of directing light traveling from the core in a radial direction toward an outer surface of the diffuser tip for irradiating a material in proximity of the diffuser tip, the pattern of light directing features having a proximal portion, a central portion and a distal portion, wherein a dimensional property of light directing features located in at least one of the proximal and distal regions is different than the dimensional property of light directing features located in the central region, the dimensional property being selected to provide a flattened light intensity profile during use.

14. The method of claim 13 further comprising connecting the optical fiber to a source of light energy.

15. The method of claim 13 further comprising placing optical coupling material between the core and the diffuser tip such that the optical coupling material and surfaces of the shaped recesses form boundaries upon which light rays traveling from the core are incident.

16. The method of claim 13, wherein the step of providing the diffuser tip with the multiple light directing features includes forming the light directing features as shaped recesses extending radially from the inner surface of the diffuser tip, the dimensional property being recess depth such that recess depths of light directing features located in the proximal and distal regions are different than recess depths of light directing features located in the central region.

17. The method of claim 13, wherein the step of providing the diffuser tip with the multiple light directing features includes forming the light directing features as shaped recesses extending radially from the inner surface of the diffuser tip and the dimensional property being periodicity of the light directing features such that the periodicity of light directing features located in the proximal and distal regions is different than the periodicity of light directing features located in the central region.

18. The method of claim 13, wherein the step of providing the diffuser tip with the multiple light directing features includes forming the light directing features as shaped recesses extending radially from the inner surface of the diffuser tip and the dimensional property is axial width of the recesses such that axial widths of light directing features located in the proximal and distal regions are different than axial widths of light directing features located in the central region.

19. The method of claim 13, wherein the step of providing the diffuser tip with the multiple light directing features includes forming the light directing features as shaped recesses extending radially from the inner surface of the diffuser tip, wherein at least one surface of each shaped recess is disposed at an angle with respect to an elongated axis of the diffuser tip, the angles of the surfaces of light directing features located in the proximal and distal regions being different than the angles of the surfaces of light directing features located in the central region.

20. The method of claim 13, wherein the step of providing the diffuser tip with the multiple light directing features includes forming shaped recesses such that one or more of the shaped recesses is substantially triangular in cross-section.

21. The method of claim 13, wherein the step of providing the diffuser tip with the multiple light directing features includes forming shaped recesses such that one or more of the shaped recesses is substantially rectangular in cross-section.

22. The method of claim 13, wherein the step of providing the diffuser tip with the multiple light directing features includes forming ring-shaped grooves that are spaced-apart axially from each other.

23. The method of claim 13, wherein the step of providing the diffuser tip with the multiple light directing features includes forming a continuous, spiral groove.
